(19) United States
(12) Patent Application Publication
(10) Pub. No.: US 2024/0130837 A1
(43) Pub. Date: Apr. 25, 2024

(54) IMPLANTABLE MEDICAL DEVICE COMPRISING A ZIRCONIA LAYER, AND METHOD FOR THE PREPARATION THEREOF

(71) Applicants: CENTRE NATIONAL DE LA RECHERCHE SCIENTIFIQUE, Paris (FR); UNIVERSITÉ DE LORRAINE, Nancy (FR); CENTRE HOSPITALIER RÉGIONAL ET UNIVERSITAIRE DE NANCY, Nancy (FR)

(72) Inventors: Pierre BRAVETTI, Nancy (FR); Richard KOUITAT-NJIWA, Laneuveville-Devant-Nancy (FR); Gaël PIERSON, Laxou (FR); Aurélien DIDELOT, Vandoeuvre-Lès-Nancy (FR)

(21) Appl. No.: 18/278,120

(22) PCT Filed: Feb. 22, 2022
(86) PCT No.: PCT/EP2022/054399

§ 371 Date: Aug. 20, 2023

(30) Foreign Application Priority Data
Feb. 25, 2021 (EP) 21305226.9

(51) Int. Cl. A61C 8/00; A61C 13/00; C04B 35/48

(52) U.S. Cl. CPC A61C 8/0013; A61C 13/001; C04B 35/48; C04B 2235/3246; C04B 2235/95; C04B 2235/963; C23C 14/083

(57) ABSTRACT

The present invention relates to an implantable medical device comprising a layer consisting of or comprising zirconia, and to a method for the preparation thereof.

The present invention relates to an implantable medical device comprising a layer consisting of or comprising zirconia, and the method for preparation thereof.

Dental implants are artificial roots which are embedded in the bone, and which are intended to create an anchoring capable of receiving a removable or fixed dental prosthesis.

Prostheses on implants are more comfortable and discreet that removable prostheses. They moreover preserve the jawbone and keep the existing tooth healthy. The placement of a bridge, on the contrary, requires sizing the adjacent teeth to serve it as a support, therefore a part of the enamel. Another disadvantage of the bridge is that the bone around the missing tooth will be progressively absorbed. Finally, with respect to a removable prosthesis, a better comfort is noted as regards the implants, a better functionality, a stability and a normal chewing, as well as a feeling of belonging.

The history of dental implants actually stated in the 1950s, when the Swedish professor, Per Ingvar Brånemark, considered as the father of modern implantology, discovered the exceptional affinity of titanium for the living bone. Titanium has thus become the first known material which is totally biocompatible. Professor Brånemark thus decided to use titanium to treat toothless patients, developing, at the time, a titanium screw which was fixed in the jaw to serve as a support for a dental prosthesis.

Upon contact with titanium, the bone will heal and be welded: it is the phenomenon of osseointregration which takes 2 to 6 months according to clinical cases. The dental implant becomes operational. It is used either as a support for a fixed prosthesis (bridge or crown) or to stabilise a removable prosthesis.

Before using titanium was developed, dental implants could not be integrated with the jawbone, as this is made possible today.

However, like all metals, titanium is not inalterable in the presence of a biological medium such as saliva, and the products coming from its electrochemical corrosion can prove to be toxic, and lead to immediate or delayed allergic reactions, which could explain the successive failures of dental implants which have occurred in certain patients. Indeed, potential hypersensitivity to titanium implants is currently a recognised fact. It is observed that a significant part of the population is sensitive to the presence of metal in the mouth. This sensitivity is conveyed by various symptoms, such as headaches, skin irritation, alteration of taste, joint problem or also chronic fatigue. A number of health problems linked to implants have been attributed to oxidation and/or corrosion events which occur, due to the biological environment, the fatigue of materials, mechanical stresses, electrogalvanism, exposure to the aggressive oral environment, concentration which are locally high in fluorides, as is the case with the application of fluorinated gels, wear of materials, or a combination of all these factors.

More recently, zirconia implants have appeared. The electrochemical problem which is due to titanium does not exist with zirconium oxide ceramic implants. Zirconia is an inert bioceramic which has excellent biomechanical properties, transmits less heat than titanium, induces no galvanic reaction, and contrary to titanium, is not sensitive to corrosion in the oral environment. In the absence of free electrons, zirconium oxide ceramics are electrical insulators and therefore are totally exempt of metal. Zirconia not being a thermal conductor means that the implants can be ground in the mouth without risk of causing necrosis of the bone. Its white colour favours aesthetic restorations. Zirconium oxide ceramic is this extremely biocompatible and seems to have no impact on the immune system. In addition, with zirconia, bacterial plaque is not deposited and this favours hygiene and the longevity of the implant-supported restoration.

However, titanium remains superior than zirconia from a mechanical standpoint, it is a ductile material with a tenacity which is six times greater. Its rupture limit is broadly greater than zirconia. Furthermore, the price of zirconia implants remains currently greater than that of conventional titanium implants.

The invention therefore aims to propose implantable medical devices, in particular dental implants, having an excellent bio- and immuno-compatibility while preserving a very good osseointegration and advantageous mechanical properties.

Another aim of the invention is to provide a method for preparing such implantable medical devices, easy to implement, without significant modifications with respect to methods of obtaining implants on the market.

Also, another aim of the invention is to provide a method for preparing such implantable medical devices, which does not modify the target roughness of the implants on the market.

Thus, according to a first aspect, the invention relates to an implantable medical device comprising, on all or some of its surface, a layer consisting of or comprising zirconia, said layer having a thickness of between around 50 and around 2000 nm.

Surprisingly, the layer consisting of or comprising zirconia of implantable medical devices of the invention, with its specific thickness, does not modify or barely modifies the roughness of said devices. This layer further has an excellent adherence to the surface of the devices and confers to said devices, the advantageous properties of zirconia, for example, its desirable visual appearance.

According to a particular embodiment, zirconia is not in the presence of, or in the form of an alloy comprising, further to zirconia, aluminium, titanium and/or carbon, zirconia not being, in particular, in the form of a zirconia-aluminium alloy, for example an ATZ, zirconia-titanium or zirconia-carbon alloy.

According to a particular embodiment, said layer has a thickness of between around 100 or 200 and around 2000 nm, in particular between around 500 or 600 and around 800 nm.

According to a particular embodiment, the invention relates to a device such as defined above, comprising a layer consisting of or comprising zirconia, said layer having on a part of the surface of said device, a thickness of between around 50 and around 2000 nm, in particular between around 50 and around 1200 nm, and on another part of the surface of said device, a thickness of between around 50 and around 2000 nm, in particular between around 800 and around 2000 nm.

According to a particular embodiment, zirconia is yttria zirconia.

According to a particular embodiment, zirconia is partially or totally in its quadratic, cubic or monoclinic phase, in particular monoclinic phase.

According to a particular embodiment, the layer comprises, further to zirconia, at least one element or compound comprising an element chosen from among Mg, Ca, Ag, and Zn, in particular in metal or oxide form.

The at least one element or compound comprising an element chosen from among Mg, Ca, Ag, and Zn can be within the layer comprising zirconia, and/or on the surface of the layer comprising zirconia. In this last case, the layer according to the invention is composed of a first sublayer comprising zirconia and of a second sublayer of said at least one element or compound comprising an element chosen from among Mg, Ca, Ag, and Zn, in particular on all or some of the sublayer comprising zirconia.

According to a particular embodiment, the element chosen from among Mg, Ca, Ag, and Zn, is at least partially on the surface of the layer, in particular over 1 to 100%, for example over 10 to 30%, of said surface.

According to a particular embodiment, the invention relates to a device such as defined above, which, in particular on its surface, excluding said layer, has no zirconia or a compound comprising it.

According to a particular embodiment, the invention relates to a device such as defined above, which, in particular its surface, consists of or comprises titanium or aluminium.

According to a particular embodiment, titanium is grade 4 titanium, or in the form of an alloy, in particular grade 5 titanium or a titanium-niobium alloy.

According to a particular embodiment, titanium or the alloy containing it has been machined, in particular cold-drawn, moulded, sintered, printed by additive manufacture.

According to a particular embodiment, said layer has a roughness Ra of 0 to 4.0 μm, for example of 0.01 or 0.02 to 4.0 μm, in particular of 0.6 to 2.0 μm, in particular of 1.0 to 1.5 μm.

The device such as mentioned above can be any implantable medical device well-known to a person skilled in the art.

According to a particular embodiment, said device is an implant, in particular a dental implant, or a prosthesis, in particular a hip prosthesis, more specifically a cupula, a shoulder prosthesis, or a knee prosthesis.

According to a particular embodiment, said device is a dental implant comprising a bone contact zone, of which all or some of the surface is covered with said layer with a first thickness of between around 50 and around 2000 nm, in particular between around 50 and around 1200 nm, and a gum contact zone, of which all or some of the surface is covered with said layer with a second thickness of between around 50 and around 2000 nm, in particular between around 800 and around 2000 nm.

According to another aspect, the invention also relates to a method for preparing an implantable medical device comprising, on all or some of its surface, a layer consisting of or comprising zirconia, said layer having a thickness of between around 50 and around 2000 nm, said method comprising a deposition step, over all or some of the surface of an implantable medical device, of a layer consisting of or comprising zirconia, PCT/EP2022/054399 said layer having a thickness of between around 50 and around 2000 nm, said deposition being carried out by physical vapour deposition (PVD) or by cold spray.

The deposition can be done by any physical vapour deposition or cold spray technique, well-known to a person skilled in the art.

Any particular embodiment described above regarding the device of the invention is also applied in this case, individually or in combination.

According to a particular embodiment, the physical vapour deposition is carried out by magnetron cathode spraying.

According to a particular embodiment, said implantable medical device is in rotation, or not, about at least one axis of rotation during all or some of the deposition.

According to a particular embodiment, the deposition is carried out by physical vapour deposition (PVD), in particular by magnetron cathode spraying, and is done in the presence of a zirconium or yttria zirconium target.

According to a particular embodiment, the deposition is done in the presence of a zirconium target or a target of at least one element or one compound comprising at least one element chosen from among Mg, Ca, Ag, and Zn.

According to another particular embodiment, the deposition is done in the presence of a zirconium target, and by bringing the device into contact with at least one element and one compound comprising at least one element chosen from among Mg, Ca, Ag, and Zn, by electrophoresis or by plasma jet.

According to a more particular embodiment, said at least one element or at least one compound comprising at least one element chosen from among Mg, Ca, Ag, and Zn is introduced, in particular in the form of nanoparticles, in the plasma jet formed during the zirconia deposition.

According to another more particular embodiment, said at least one element or one compound comprising at least one element chosen from among Mg, Ca, Ag, and Zn is deposited together with zirconia.

Also, according to another more particular embodiment, said at least one element or one compound comprising at least one element chosen from among Mg, Ca, Ag, and Zn is deposited after zirconia has been deposited.

According to a particular embodiment, the variation between the roughness Ra of the initial device and the roughness Ra of the device obtained from the method is less than or equal to around 25%, for example less than or equal to around 20, 15, 10, 9, 8, 7, 6, 5, 4, 3, 2 or 1%.

According to another particular embodiment, the variation between the roughness Ra of the initial device and the roughness Ra of the device obtained from the method is less than or equal to around 1%, for example less than or equal to around 0.9; 0.8; 0.7; 0.6; 0.5; 0.4; 0.3; 0.2; or 0.1%.

According to a particular embodiment, the deposition step is preceded by a stripping step, in particular plasma stripping, of the device.

This step can make it possible, if necessary, to increase the adherence of the layer consisting of or comprising zirconia. The implantable medical device can be obtained by any technique well-known to a person skilled in the art.

For example, the implantable medical device, in particular a dental implant, can be obtained by machining, in particular cold-drawing, moulding, sintering, printing by additive manufacture, or any other ad hoc technique well-known to a person skilled in the art.

According to another aspect, the invention relates to an implantable medical device which can be obtained according to the method such as defined above.

### Definitions

Such as is understood in this case, the value ranges in the form of “x-y”, or “from x to y”, or “between x and y”, include the limits x and y as well as integers comprised between these limits. As an example, “1-5”, or “from 1 to 5”, or “between 1 and 5”, mean the integers 1, 2, 3, 4 and 5. The preferred embodiments each include an integer taken individually in the value range, as well as any subcombination of these integers. As an example, the preferred values for “1-5” can comprise the integers 1, 2, 3, 4, 5, 1-2, 1-3, 1-4, 1-5, 2-3, 2-4, 2-5, etc.

Such as used in this case, the term “around” refers in particular to a value range at ±10% of a specific value. For example, the term “around 100” comprises the values of 100±10%, i.e. the values of 90 to 110.

By “zirconia”, this means in particular, zirconium dioxide, also known under the name of zirconium oxide (IV), or of compound of formula ZrO₂.

By “roughness Rₐ”, this means in particular the average distance between the average line and the peaks and the cavities of a given surface. Therefore, this is, in particular, for a given surface, of the mean deviation, or arithmetic mean of the distances between successive peaks and cavities. As an example, the evaluation length of the roughness can be 1.25 mm (corresponding, in particular, to a high-pass filter cutoff wavelength of the measuring appliance of 0.25 mm), in particular for a roughness Rₐ such that 0.02<Ra 0.1; of 4.00 mm (corresponding, in particular, to a high-pass filter cutoff wavelength of the measuring appliance of 0.80 mm), in particular for a roughness Rₐ such that 0.1<Ra<2; or of 12.50 mm (corresponding in particular to a high-pass filter cutoff wavelength of the measuring appliance of 2.50 mm), in particular for a roughness Rₐ such that 2<Ra<10.

“Ra” thus corresponds in particular to the difference between this mean distance and the “central line”. More specifically, the roughness Rₐ is measured according to the standard ISO 4287, for example on a Dektak Stylus® (Bruker) profilometer.

### EXAMPLES

### Example 1: Preparation of an Implantable Medical Device According to the Invention

An implantable medical device on the market, for example, a dental implant, is prepared according to the following steps:
cleaning with alcohol (pure ethanol);
bathing in an ultrasonic tank with water, for example distilled water;
drying by pure air blowing;
installing the device in the PVD machine;
if necessary, cleaning of the device by pure air blowing;
vacuuming of the PVD machine;
if necessary, plasma stripping of the device.

Once the device is prepared as indicated above, the layer consisting of or comprising zirconia is deposited on said device as follows:
cleaning of the PVD machine (in particular sandblasting the substrate carrier, screen suppressors, etc.);
implementing the zirconium target, optionally yttria;
implementing the device on the rotary substrate carrier;
vacuuming the machine (for example, 2 to 5.10⁻⁶ mbar);
introducing plasma gas (for example, 30 sccm of argon);
introducing reactive gas (for example, 6 sccm of oxygen);
adjusting the pressure inside the deposition chamber using the butterfly valve (for example, at 0.75 Pa);
switching on the pulsed DC generator (Power=0.5 A−Frequency=100 kHz−Idle time=2 μs, for example);
if necessary: priming the plasma using an RF generator;
deposition time to be adapted according, in particular, to the thickness of the desired deposition layer (for 100 nm, 1 hour of deposition can be necessary, for example, on a non-industrial machine. For 800 nm, around 8 hours of deposition can be necessary);
end of deposition (by stopping the generator and gas flow, in particular);
stopping of the molecular turbo pump and of the primary pump;
purging of the development chamber;
recovery of the device according to the invention, preferably with gloves.

The parameters indicated above are given as examples and can be adapted easily by a person skilled in the art, according to the PVD machine used.

In particular, devices of the invention, have been obtained according to the present example, with a zirconia thickness of 100 nm or of 800 nm, from dental implants on the market:

| Name of the | | Thickness |
|---|---|---|
| device of the | Reference of the implant used | of the zirconia |
| invention | which is on the market | layer |
| A100 | Naturall + of the company ETK ® | 100 nm |
| A800 | Naturall + of the company ETK ® | 800 nm |
| B100 | Natea + of the company ETK ® | 100 nm |
| B800 | Natea + of the company ETK ® | 800 nm |

Each of the 4 implants above have been completely covered with zirconium oxide over their entirety, by magnetron cathode spraying. To do this, the target corresponded to pure zirconium in metal form. The gas present was oxygen and the substrate was the implant to be sprayed.

Thus, by ion bombardment on the zirconium target, a zirconium metal vapour is formed, which is oxidised by the oxygen present during its route to the implant. Zirconium oxide is thus fixed to the implant.

### Example 2: Tearing Tests Carried Out on the Devices of the Invention

Two devices of the invention obtained according to example 1, one with a zirconia thickness of 100 nm, the other with a zirconia thickness of 800 nm, have been scratched in the form of a check pattern, using a diamond comb. The scratches are spaced apart by around 750 μm.

A piece of adhesive tape is adhered onto the check pattern formed, then torn.

The check pattern is thus observed under an optical microscope.

When the check pattern observed under a microscope is intact, the test is successful. When the layer is unstuck, torn in at least one place of the check pattern, the test is not successful.

For the two devices of the invention, the zirconia layer is fully adherent after the test. No peeling off can be seen. The test is therefore successful and the adherence of the layer is greatly satisfactory as regards the devices of the invention.

### Example 3: Roughness Measurements on the Devices of the Invention

Materials and Methods

Transverse cross-sections of the devices of the invention, for example of the apical part of a dental implant according to the invention, have been made using a micro-cutting machine. Samples of each device of 4 to 6 mm of thickness have thus been obtained. Each of them has been analysed with a profilometer and with a scanning electron microscope (SEM). This makes it possible, if necessary, to keep the integrity of the devices studied.

The profilometer (or roughness meter) makes it possible to measure the deviations of 500Â to 1 mm of a surface. It is composed of a diamond stylet which is placed and is moved on the sample with a constant force. These physical data are converted into electrical information on a computer. The topography, the surface corrugation and the roughness of the sample are obtained.

Each measurement is taken over three different zones of each device sample. These measurements are 200 μm. In order to obtain a value representative of the surface roughness, the mean of the three values obtained has been calculated.

The measurement is taken on a Dektak Stylus® (Bruker) profilometer according to the standard ISO 4287.

The scanning electron microscope (SEM) makes it possible to take atomic composition measurements of the devices from the samples. The composition measurements are taken by energy dispersion X-ray spectroscopy or energy dispersive spectroscopy (EDS) by X-ray emission. The impact of the electron beam on the sample produces X-rays characteristic of the elements present on the sample. A qualitative composition measurement spectrum of the sample is obtained, also with the three-dimensional image acquisition.

Results

The roughness of the devices A, B, C and D of the invention, mentioned in example 1 has been measured as indicated above.

A reference implant (outside of the invention), implant on the market without a zirconia layer, has also been considered.

The results obtained, corresponding to the mean of the three measurements, as indicated above, show that the variation between the roughness Ra of the initial device and the roughness Ra of the device obtained from the method is less than or equal to around 25%, in particular less than or equal to around 20%, in particular less than or equal to around 7%.

1. Implantable medical device comprising, over all or some of its surface, a layer comprising zirconia, said layer having a thickness of between around 50 and 2000 nm, wherein the layer comprises, further to zirconia, at least one element or compound comprising an element chosen from among Mg, Ca, Ag, and Zn, in particular in metal or oxide form, located at least partially on the surface of the layer.

2. Device according to claim 1:
wherein said layer has a thickness of between around 100 or 200 and around 2000 nm, in particular between around 500 or 600 and around 800 nm; or
comprising a layer comprising zirconia, said layer having, over a part of the surface of said device, a thickness of between around 50 and around 2000 nm, in particular between around 50 and around 1200 nm, and over another part of the surface of said device, a thickness of between around 50 and around 2000 nm, in particular between around 800 and around 2000 nm.

3. Device according to claim 1, wherein zirconia is yttria zirconia, and/or partially or totally in its quadratic, cubic or monoclinic phase, in particular monoclinic.

4. Device according to claim 1, wherein the at least one element or compound comprising an element chosen from among Mg, Ca, Ag, and Zn, is located at least partially on the surface of the layer, over 10 to 30% of said surface.

5. Device according to claim 1, which, in particular its surface, consists of or comprises titanium or aluminium, titanium being in particular grade 4 titanium, or in the form of an alloy, in particular grade 5 titanium or a titanium-niobium alloy.

6. Device according to claim 1, wherein said layer has a roughness Ra of between 0 and 4.0 μm, in particular of 0.6 to 2.0 μm, in particular of 1.0 to 1.5 μm.

7. Device according to claim 1, which is an implant, in particular a dental implant, or a prosthesis, in particular a hip prosthesis, more specifically a cupula, a shoulder prosthesis, or a knee prosthesis.

8. Method for preparing an implantable medical device according to claim 1, comprising a step of depositing, over all or some of the surface of an implantable medical device, a layer comprising zirconia, said layer having a thickness of between around 50 and around 2000 nm, said deposition being carried out by physical vapour deposition (PVD); in the presence of a zirconium target and a target of at least one element or one compound comprising at least one element chosen from among Mg, Ca, Ag, and Zn; or in the presence of a zirconium target, and by bringing the device into contact with at least one element or one compound comprising at least one element chosen from among Mg, Ca, Ag, and Zn, by electrophoresis or by plasma jet, said at least one element or at least one compound comprising at least one element chosen from among Mg, Ca, Ag, and Zn being introduced, in particular in the form of nanoparticles, in the plasma jet formed during the zirconia deposition.

9. Method according to claim 8, wherein the physical vapour deposition is carried out by magnetron cathode spraying.

10. Method according to claim 8, wherein said implantable medical device is in rotation, or not, about at least one axis of rotation during all or some of the deposition.

11. Method according to claim 8, wherein said at least one element or one compound comprising at least one element chosen from among Mg, Ca, Ag, and Zn is deposited:
together with zirconia; or
after zirconia has been deposited.

12. Method according to claim 8, wherein the variation between the roughness Ra of the initial device and the roughness Ra of the device obtained from the method is less than or equal to around 25%.
